(19) United States
(12) Patent Application Publication
(10) Pub. No.: US 2023/0321288 A1
(43) Pub. Date: Oct. 12, 2023

(54) RIBOFLAVIN PHOTOCHEMICAL TREATMENT (RPT)-BASED INACTIVATION METHOD OF PATHOGENS IN BIOLOGICAL LIQUID SAMPLE

(71) Applicant: INSTITUTE OF BLOOD TRANSFUSION CHINESE ACADEMY OF MEDICAL SCIENCES, Chengdu, Sichuan (CN)

(72) Inventors: Zhong LIU, Chengdu, Sichuan (CN); Yundi YIN, Chengdu, Sichuan (CN); Ling LI, Chengdu, Sichuan (CN); Haixia XU, Chengdu, Sichuan (CN); Li GONG, Chengdu, Sichuan (CN)

(73) Assignee: INSTITUTE OF BLOOD TRANSFUSION CAMS, Chengdu, Sichuan (CN)

(21) Appl. No.: 18/034,643

(22) PCT Filed: Oct. 27, 2021
(86) PCT No.: PCT/CN2021/126678

§ 371 Date: Apr. 28, 2023

(30) Foreign Application Priority Data
Oct. 30, 2020 (CN) 202011197064.7

(51) Int. Cl. A61L 2/00

(52) U.S. Cl. CPC A61L 2/0047; A61L 2/0088; A61L 2101/44

(57) ABSTRACT

The present disclosure relates to a riboflavin photochemical treatment (RPT)-based inactivation method of pathogens in a biological liquid sample. Aiming at the problems existing in the current riboflavin-based pathogen inactivation methods, a technical solution of the present disclosure is to provide an RPT-based inactivation method of pathogens in a biological liquid sample, including the following steps: adding riboflavin to a biological liquid sample to be treated, and conducting irradiation on the biological liquid sample with light; where the light is narrow-spectrum ultraviolet (UV) light with a wavelength of 360 nm to 370 nm and/or 390 nm to 400 nm. In the present disclosure, parameters such as an irradiation time, an irradiation intensity, and a riboflavin concentration are further optimized. The inactivation method can achieve an excellent pathogen inactivation effect, and has little damage to other components in the biological liquid sample.

### TECHNICAL FIELD

The present disclosure belongs to the technical field of pathogen inactivation, and in particular relates to a riboflavin photochemical treatment (RPT)-based inactivation method of pathogens in a biological liquid sample.

### BACKGROUND

In medicine or biology, pathogens are generally present in biological liquid samples and need to be inactivated. For example, blood-borne pathogens, including viruses, bacteria, protozoa, and spirochetes, are inevitably introduced during the collection and transfer of blood products.

With the development of blood pathogen detection technology, the risk of blood transfusion infection has been greatly reduced. However, the pathogen detection window still exists, and current routine detection methods cannot cover all known blood-borne pathogens. Emerging and re-emerging blood-borne pathogens still threaten the safety of blood transfusion. In particular, platelets (PLTs) are routinely stored at 20° C. to 24° C., and have a higher risk of bacterial contamination. Accordingly, continuous and stable blood-borne bacterial contamination has become the most threatening factor to blood supply institutions.

The development of pathogen inactivation technology can effectively reduce the risk of blood transfusion infection. So far, the promising blood pathogen inactivation technologies mainly include psoralen and riboflavin, which can be used to inactivate pathogens in plasma, platelets, and red blood cells. The psoralen may be genotoxic and therefore need to be removed after use. In contrast, the riboflavin (vitamin B2) is an essential natural vitamin for the human body, and its decomposition products are widely present in the blood and tissues of the human body. Moreover, the riboflavin has a natural photochemical reaction and does not need to be removed after use. As a result, riboflavin is widely used in the inactivation of pathogens in blood components.

For example, the Chinese patent “CN201910975223.2, Equipment and method for inactivating pathogens in blood components by RPT” proposed a method for inactivating pathogens in blood by RPT, and the conditions for inactivation were preferred. In this patent, the preferred conditions disclosed were narrow-band UV light at 309 nm to 313 nm, with an optimal illumination time of 5 min to 40 min and an optimal illumination energy range of 0.4 J/ml to 3 J/ml. However, under these conditions, there is still a poor inactivation effect on pathogens, and there are still some damages to other components in the blood products. Based on the above reasons, although the riboflavin pathogen inactivation system is used in European and other countries to inactivate pathogens in blood, this system has not yet been approved by the FDA in the United States.

### SUMMARY

Aiming at the problems existing in the current riboflavin pathogen inactivation methods, the present disclosure provides a riboflavin photochemical treatment (RPT)-based inactivation method of pathogens in a biological liquid sample. A purpose of the present disclosure is as follows: by optimizing a wavelength range of light irradiation, the riboflavin photochemical inactivation method has a better inactivation effect on pathogens and lower damage to blood components.

The present disclosure provides a RPT-based inactivation method of pathogens in a biological liquid sample, including the following steps: adding riboflavin to a biological liquid sample to be treated, and conducting irradiation on the biological liquid sample with light; where the light is narrow-spectrum UV light with a wavelength of 360 nm to 370 nm and/or 390 nm to 400 nm.

Preferably, the light is the narrow-band UV light with a wavelength of 390 nm to 400 nm.

Preferably, the light is UV light with a peak at 395 nm.

Preferably, the irradiation is conducted on the biological liquid sample with the light for 3 min to 30 min at a light energy range of 0.2 J/ml to 5 J/ml.

Preferably, the irradiation is conducted on the biological liquid sample with the light for 3 min to 4.9 min at a light energy range of 0.2 J/ml to 0.39 J/ml.

Preferably, 40 μM to 60 μM of the riboflavin is added to the biological liquid sample.

Preferably, 50 μM of the riboflavin is added to the biological liquid sample.

Preferably, the biological liquid sample is selected from the group consisting of a blood product, a cell product, or a tumor cell sample.

Preferably, the blood product is selected from the group consisting of whole blood, leukoreduced whole blood, packed red blood cells, manual platelets, apheresis platelets, plasma, and cryoprecipitate.

In the present disclosure, the RPT-based inactivation method of pathogens in biological liquid samples (such as blood products) by riboflavin is improved, and the wavelength of light irradiation is optimized. Under the same irradiation time and irradiation intensity, the light irradiation in the preferred wavelength range of the present disclosure has a better inactivation effect on the pathogens. In addition, light irradiation in this wavelength range has a better inactivation effect on pathogens. Therefore, in actual operations, narrow-band UV light in this wavelength range is selected to inactivate pathogens in biological liquid samples under shorter illumination time and lower illumination intensity. In this way, light damages to other components in the biological liquid sample are reduced.

In the present disclosure, the inactivation method is suitable for the inactivation of pathogens in blood products, the decontamination of various biological liquid samples, and the treatment of patients with clinical severe infections and tumors, and has wide application prospects.

Obviously, according to the above-mentioned content of the present disclosure, other various forms of modification, substitution or change can also be made based on the common technical knowledge and conventional means in the art without departing from the above-mentioned basic technical idea of the present disclosure.

The above-mentioned content of the present disclosure will be further described in detail below through the specific implementation in the form of examples. However, they should not be construed as limiting the scope of the above-mentioned subject of the present disclosure to the following examples. All technologies implemented based on the above-mentioned content of the present disclosure fall within the scope of the present disclosure.

### BRIEF DESCRIPTION OF THE DRAWINGS

FIG. 1 shows inactivation effects of narrow-band UV light in different wavelength ranges on Escherichia coli in the RPT-based inactivation method of pathogens in blood products; and

FIG. 2 shows inactivation effects of narrow-band UV light with a wavelength of 395 nm±5 nm and narrow-band UV light with a wavelength range of 309 nm to 313 nm on Staphylococcus aureus in platelets.

### DETAILED DESCRIPTION

The technical solutions of the present disclosure are further described below with reference to specific examples.

In the present disclosure, specific operations and devices of the RPT-based inactivation method of pathogens in a biological liquid sample can be conducted with reference to contents disclosed in the prior art. In the following examples, the specific operations and devices used were consistent with the methods and devices disclosed in the Chinese patent “CN201910975223.2, Equipment and method for inactivating pathogens in blood components by RPT”. The difference was that the wavelength range of light irradiation, irradiation time, irradiation energy, and riboflavin concentration had been changed.

### Example 1

In this example, the plasma of healthy blood donors containing 50 μM of riboflavin was irradiated with LED lamp beads in a series of wavelength ranges for 15 min at an irradiation intensity of 1 W. The growth of E. coli in the blood products was then detected by a Reed-Muench method.

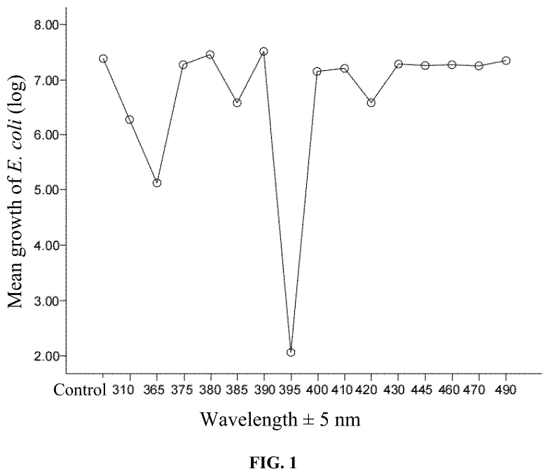

The results were shown in FIG. 1. Under the same irradiation time and irradiation intensity, in a series of narrow-band UV light, LED lamp beads with a wavelength of 365 nm f 5 nm and a wavelength of 395 nm±5 nm had a desirable pathogen inactivation effect on E. coli in the RPT-based inactivation system.

### Example 2

In this example, the apheresis platelets containing 50 μM of riboflavin were separately irradiated with LED lamp beads with a wavelength of 395 nm±5 nm and fluorescent tubes with a wavelength of 309 nm to 313 nm for 30 min at irradiation intensities of 1 W (395 nm f 5 nm) and 9 W (309 nm to 313 nm), respectively. The growth of Staphylococcus aureus in the blood products was then detected by a Reed-Muench method.

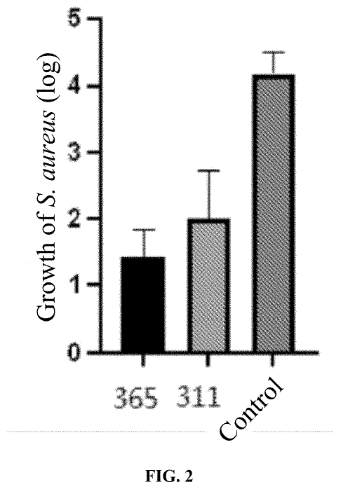

The results were shown in FIG. 2, and the data of a control experimental group without RPT were also shown in this figure. It was seen from the figure that both the 395 nm±5 nm LED lamp beads and the fluorescent tubes with a wavelength of 309 nm to 313 nm could inactivate the S. aureus. However, under the same irradiation time and irradiation intensity, the inactivation effect of 395 nm±5 nm LED lamp beads was better than that of fluorescent tubes with a wavelength of 309 nm to 313 nm.

The properties and component contents of the platelet samples in the three groups of experiments were compared, and the results were as follows:

TABLE 1
Quality of platelet preservation

| | | | Control |
|---|---|---|---|
| Test item | 395 nm ± 5 nm | 309 nm to 313 nm | (no irradiation) |
| PH | 7.45 ± 0.01 | 7.26 | 7.53 |
| Na⁺ mmol/L | 153.6 ± 0.49 | 151 | 152 |
| K⁺ mmol/L | 2.7 ± 0 | 3 | 2.6 |
| Glu mmol/L | 26.3 ± 0.09 | 23.6 | 26 |
| Lac mmol/L | 9.24 ± 0.08 | 10.6 | 8.1 |
| HCO₃-mmol/L | 8.54 ± 0.22 | 8.5 | 10 |
| HCO₃ std | 15.26 ± 0.15 | 12.2 | 17.2 |
| TCO₂ | 8.94 ± 0.22 | 9.1 | 10.4 |
| PLT | 714.2 ± 6.49 | 297 | 717 |
| PDW | 10.84 ± 0.16 | 17.3 | 10.9 |
| MPV | 9.5 ± 0 | 12.1 | 9.4 |
| P-LCR | 21.2 ± 0.2 | 36.7 | 20.7 |
| PCT | 0.68 ± 0.01 | 0.36 | 0.68 |

From the data in Table 1, it was seen that after inactivating pathogens on platelets by RPT, the parameters of various properties and component contents deviated from those of the control group without light. However, an irradiation effect of the 395 nm±5 nm LED lamp beads on the various properties and component contents was significantly smaller than an irradiation effect of the 309 nm to 313 nm fluorescent tubes on the various properties and component contents. This showed that the light irradiation of 395 nm±5 nm had significantly less damage to the platelet samples.

### Example 3

In this example, the light dose of 309 nm to 313 nm narrow-band UV light and the light dose of 395 nm±5 nm narrow-band UV light were compared under a same inactivation effect.

The specific implementation steps were as follows:

1. 10 μL of a S. aureus culture solution was added to 150 ml of a bag of plasma from a healthy blood donor (supported by the local ethics committee), to obtain a bacterial plasma suspension of about 4 log to 5 log.

2. 9 mL of the bacterial plasma suspension was mixed with 1 mL of physiological saline as a control, and placed in a refrigerator at 4° C.

3. A 500 μmon riboflavin-containing physiological saline (CAS: 83-88-5; purchased from Sigma-Aldrich, St. Louis, Missouri, USA) was added to the bacterial plasma suspension, such that a final riboflavin concentration was 50 μmol/L.

4. 300 μL of a riboflavin-added bacterial plasma suspension was transferred to a sterile 24-well plate (with a well diameter of 1.5 cm), and then separately exposed to 9 W 309 nm to 313 nm fluorescent tubes (UVB narrow-band PL-L/PL-S, Philips, Amsterdam, The Netherlands) and 1 W 395 nm±5 nm LED lamp beads for irradiation under a temperature-controlled environment (20° C. to 24° C.).

The light dose of the 309 nm to 313 nm fluorescent tubes was 9.76 J/mL, and an irradiation time was 30 min. The light dose of the 395 nm±5 nm LED lamp beads was 1.25 J/mL, and an irradiation time was 10 min. The two experimental samples were conducted in parallel for 6 groups.

5. After the irradiation was over, the experimental sample and the control sample were serially diluted by 101 to 106.

6. 100 μL of each diluted sample was added to a center of a sterile plate, the inoculation of each diluted sample was repeated 8 times, and the bacterial growth was determined.

7. After culturing in a 37° C. biochemical incubator (SHP-080, Jinghong, China, Shanghai) for 24 h to 48 h, the growth of bacteria in each well was observed and recorded, and a bacterial titer was calculated using a Reed-Muench method.

The experimental results showed that after irradiating with 309 nm to 313 nm UV light at a higher light dose and using 395 nm±5 nm UV light at a lower light dose, the bacteria growth at 309 nm to 313 nm and 395 nm±5 nm were 2.01 log±1.99 log and 2.22 log±1.80 log, respectively. There was no statistical difference between the two (P=0.568), that is, the inactivation effect on pathogens was equivalent.

It was seen from the above examples that the narrow-band UV light in the preferred wavelength range of the present disclosure had a better inactivation effect on pathogens and less damages to other components in biological liquid samples (such as blood products). In addition, using the narrow-band UV light in the preferred wavelength range of the present disclosure could select shorter irradiation time and lower irradiation energy, thereby further reducing the damages of light irradiation to other components in biological liquid samples (such as blood products).

1. A riboflavin photochemical treatment (RPT)-based inactivation method of pathogens in a biological liquid sample, comprising the following steps: adding riboflavin to a biological liquid sample to be treated, and conducting irradiation on the biological liquid sample with light; wherein the light is narrow-spectrum ultraviolet (UV) light with a wavelength of 390 nm to 400 nm;
the irradiation is conducted on the biological liquid sample with the light for 10 min to 30 min at a light energy range of 0.2 J/ml to 5 J/ml and an ambient temperature of 20° C. to 24° C.; and 40 μM to 60 μM of the riboflavin is added to the biological liquid sample; and
the biological liquid sample is a blood product.

2.-9. (canceled)

10. The RPT-based inactivation method of pathogens in a biological liquid sample according to claim 1, wherein the light is UV light with a peak at 395 nm.

11. The RPT-based inactivation method of pathogens in a biological liquid sample according to claim 1, wherein the blood product is selected from the group consisting of whole blood, leukoreduced whole blood, packed red blood cells, manual platelets, apheresis platelets, plasma, and cryoprecipitate.
